(19) United States
(12) Patent Application Publication
(10) Pub. No.: US 2005/0159671 A1
(43) Pub. Date: Jul. 21, 2005

(54) Method for diagnosing, detecting, and monitoring brain function including neurological disease and disorders

(75) Inventor: Robert Shaw Sneddon, Pasadena, CA (US)

Correspondence Address: Robert S. Sneddon, 595 North Garfield Apt. 6, Pasadena, CA, 91101, US

(73) Assignee: Sneddo & Associates Inc.

(21) Appl. No.: 11/016,332

(22) Filed: Dec. 18, 2004

Related U.S. Application Data
(60) Provisional application No. 60/530,337, filed on Dec. 18, 2003.

(51) Int. Cl. A61B 5/04

(52) U.S. Cl. 600/544

(57) ABSTRACT

The present invention provides a method for diagnosing, detecting and monitoring brain function, especially neurological diseases and disorders. This invention examines the output of a neurological monitoring device such as an electroencephalography (EEG) recording. The EEG recording is often taken while a person is engaged in a specific neurological task such as delayed recognition. This invention provides for two methods for the diagnosis, detection and brain monitoring based on the EEG recording. The first is the use of the person as their own baseline for comparison. The efficacy of a person's brain function is measured by comparing a portion of their EEG recording with a different portion. Each of these portions is taken from the same EEG recording of a single neurological task performance. The second method is the minimal use of monitoring device output, such as an EEG recording, in a manner congruent with the neurological task being performed by the person. For example, to test a person's delayed recognition memory; a person would first be required to perform a delayed recognition memory task. Then, the EEG recording for two electrodes, P3 and P4 would be examined for the first 150 milliseconds after recognition memory stimulus onset. Then the EEG recording for two other electrodes, T7FPp1 and T8Fp2 would be examined for the next 150 milliseconds. Since this choice of electrodes and times is congruent with the neurological task of delayed recognition, the data is highly relevant to the monitoring of the person's delayed recognition memory. This method, combined with using a person as their own baseline allows this invention to provide high accuracy in the detection, diagnosis and monitoring of brain function, especially neurological diseases and disorders.

### CROSS-REFERENCE TO RELATED APPLICATIONS

The present patent document claims the benefit of the filing date under 35 U.S.C. §119(e) of Provisional U.S. Patent Application Ser. No. 60/530,337 filed Dec. 18, 2003, which is hereby incorporated in its entirety by reference.

### BACKGROUND OF THE INVENTION

This invention relates to the detection, diagnosis and monitoring of brain function. It is especially useful for the detection, diagnosis and monitoring of neurological disorders with electroencephalography (EEG).

It is well known that neurological anomalies can be reflected in the electrical activity of the brain. Such electrical activity is therefore commonly used to diagnose a variety of neurological disorders or to evaluate the treatment thereof. Electrical activity in the brain is typically captured and analyzed in the form of an electroencephalograph (EEG).

An ERP represents neural electrical activity that occurs as a result of a specific sensory stimulus to the patient, such as a flash of light or a tone. The electrical activity, measured as voltage (that is, potential), is therefore an evoked response to a stimulus. Like an EEG, an ERP is typically collected and analyzed as a waveform. ERPs also tend to be less variable than EEGs over multiple trials on a given patient.

Still, a fundamental problem in both ERP and EEG diagnostic methods is individual variation. There is considerable variation from individual to individual in both the ERP and EEG. For this reason, there is absence of high diagnostic accuracy in many portions of neurology and psychiatry. One solution, given in U.S. Pat. No. 6,622,036 is the use of a large database, covering many individuals, to compare EEG data with. However, these data must be specific to an individual medication as well as an individual type of EEG. The result is an enormous database requirement.

Other methods such as that given in U.S. Pat. No. 6,463,321 attempt to characterize individual ERPs as a signal vector. This does not eliminate the large amount of individual variation. Instead, this signal vector must be compared to a data base of signal vectors for the purposes of diagnosis.

Overall, a fundamental difficulty in the prior art is a lack of ability to deal directly with individual EEG and ERP variations. Instead, the prior art compares EEG and ERP outcomes to other outcomes. These other outcomes might be taken from the person performing similar, but different ERP tasks. Or they might come from the person performing the same task at different times, or they might come as a comparison between different people. The fundamental aspect of the prior art is that comparisons for EEG recordings are done between recordings. The present invention compares a person's EEG or ERP to their own EEG or ERP within the performance of the same task.

### BRIEF SUMMARY OF THE INVENTION

The invention described herein provides a method of diagnosing the presence of a neurological disorder (such as Alzheimer's Disease, depression, or schizophrenia), otherwise assessing the neurological condition of a patient, or characterizing the results of a treatment regimen used by a patient. The method includes the collection and analysis of ERP data. The method of the invention begins by conducting a plurality of ERP trials on a patient. In an embodiment of the invention, the data from the ERP trials is then examined in a manner congruent with psychophysical task being performed when the ERP data is collected. This greatly increases the relevance of the data to the analysis and reduces the artifacts in the data. Typical congruence analysis consists of examining the ERP data at the times and scalp locations (electrode positions) which correspond to the neural activity engendered by the psychophysical task. Measures of ERP activity over a relatively small number of electrodes, e.g., two, and a relatively small time period, e.g., 150 milliseconds are used to characterize the condition of the person. Comparisons of ERP activity at different times and scalp locations results in characterizing a person's brain/neurological function without comparison of the person's ERP data to other individuals' ERP data. The present invention deals with the difficulty of individual variation by the use of the person's own ERP and EEG data as a baseline for comparison. Another method is using a small number of EEG electrodes for a small amount of the total time of the EEG recording.

In the prior art, quantitative EEG (qEEG) often has poor accuracy in the detection, diagnosis, and monitoring of neurological conditions, especially Alzheimer's Disease and Related Disorders (ADRD). The present invention solves this problem by examining EEG activity for a small number of electrodes over small periods of time. This approach is highly unexpected, as the prior art of qEEG detection, diagnosis, and monitoring, consists of analyzing large amounts of EEG data for a plurality of EEG electrodes.

Instead, only EEG data which is congruent to the person's neurophysiology is examined. This congruence is often achieved by using the EEG data from selected times and selected electrode positions which correspond to the neurophysiological activity of a specific psychophysical task. Detection, diagnosis and monitoring are achieved by comparing the EEG activity from a small number of time periods and a small number of electrodes to the person's own EEG activity taken from a small number of time periods and a small number of electrodes which differs in some way from the first group. Often this comparison is achieved by using quantitative techniques for the comparison of the selected EEG data sets. In the prior art, comparisons of EEG activity are done between different persons or between the same person at significantly different times or between a person performing one task and that same person performing a different task.

This method has a high level of accuracy in the detection of neurological conditions such as Alzheimer's Disease (AD). Furthermore, it results in the differential diagnosis of neurological conditions by applying the method a plurality of times for a plurality of different psychophysical tasks. It has the further use of monitoring changes in neurological health, especially changes caused by medication.

### BRIEF DESCRIPTION OF THE DRAWINGS

FIG. 1 is a picture showing Dorsal and Ventral Streams for a Working Memory Task.

FIG. 2 is a Flow Chart for the Additional Embodiment for the Detection of ADRD.

FIG. 3 is a graph of qEEG Detection of Normal Aging and Mild ADRD.

FIG. 4 is a graph of results of Monitoring Medication Treatment of Alzheimer's Disease for patient 1.

FIG. 5 is a graph of results of Monitoring Medication Treatment of Alzheimer's Disease for patient 2.

FIG. 6 is a graph of results of Monitoring Medication Treatment of Alzheimer's Disease for patient 3.

FIG. 7 is a graph showing Monitoring of Omentum Transposition Surgery.

### DETAILED DESCRIPTION OF THE INVENTION AND THE PREFERRED EMBODIMENT

This invention provides for the accurate diagnosis, detection and monitoring of a brain's state, especially for neurological diseases and disorders, with two novel methods. The first method is to use of the subject as their own baseline for comparison. The second is using a minimum of neurological data. That is, we only use data which is pertinent to the disorder which we are detecting.

For example, suppose that we wanted to check the functionality of a person's neural substrates for working memory. These neural substrates are dorsolateral prefrontal cortex (DLPFC) and the cortex along the dorsal and ventral streams prior to DLPFC. First, we would have a subject perform a specific neurological task, such as a task for working memory. Second, we would examine the neurological data which corresponds to the dorsal stream cortex, the ventral stream cortex and the DLPFC. We would only look at the data which corresponds to the activation of these cortices after the onset of a working memory stimulus. This would be about 150 milliseconds (ms) for the dorsal and ventral cortices and about another 150 ms for the DLPFC. Then we could compare the activity at the DLPFC to the dorsal activity and also to the ventral activity. Thus, the person would serve as there own baseline. This comparison might be judged in the light of other people's performances, but this is not necessarily the case, as will be described later.

This use of a person as their own baseline within the performance of a neurological task is unexpected. Normally an individual's neurological activity is compared between tasks. For example, in the P300 “odd event” paradigm, a person performs several tasks where they look at the same sort of object, say a picture of a ball. Then, they suddenly look see picture of something incongruous, like an orange. This “orange viewing” event produces the desired effect, an evoked potential at 300 ms. That potential is significantly higher than the potential at 300 ms when the person is seeing pictures of a ball.

Other between tasks comparisons are made when a person's neurological performance on a task is compared to a different performance of a task, or to other people's task performance. In all cases, comparisons are made between tasks, not within tasks. For a specific example, the detection of Alzheimer's Disease and Related Disorders (ADRD) with electroencephalography (EEG) will be described.

In the preferred embodiment, subjects' EEGs are recorded while they perform delayed recognition memory tasks. These tasks consist of viewing an object or face which the subject has seen ten minutes earlier. Subjects press a yes/no button indicating whether or not they remember seeing the object or face. Given that this is a delayed recognition task, we sought to monitor subjects' EEGs in a manner which is congruent with the neurophysiological process of delayed recognition. This means examining EEG data at the times and scalp locations which correspond with the neural pathways and patterns of cortical activation of a delayed recognition task.

Neurophysiology of Delayed Recognition

Delayed recognition is associated with the hippocampal area and the parahippocampal cortices^{6,9,11,12,21,25-28}. However, the neural pathways and patterns of cortical activation of a delayed recognition task are not well understood. We assume that a delayed recognition task is related to a working memory task. This assumption is acceptable for a number of reasons. First, both afferent and efferent connections exist between the hippocampus and dorsolateral prefrontal cortex (DLPFC). Second, there is evidence that post-retrieval monitoring of a recognition memory is done by prefrontal cortex^{7,14,23,30}. And third, both lateral prefrontal cortex and the hippocampus are involved in novelty detection (P300)¹⁹.

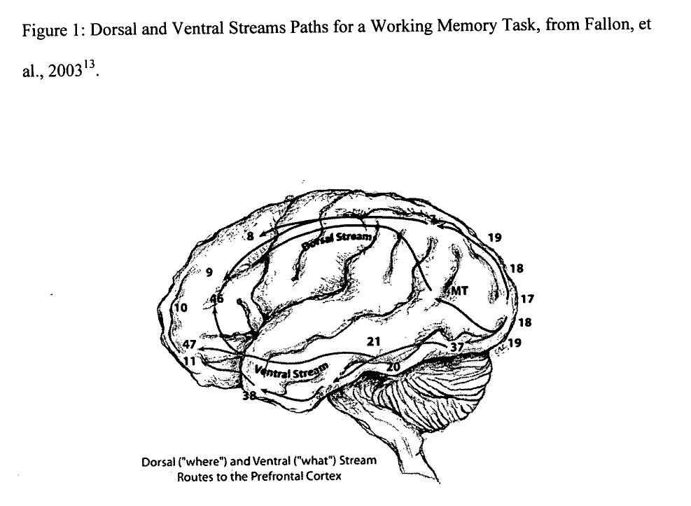

The basic neural pathway of a working memory task^{8,16,17} is diagrammed by Fallon et al.¹³ in FIG. 1. This is the dorsal and ventral stream diagram. Both of these streams begin in primary visual cortex, area 17. They follow separate paths which come back together in dorsolateral prefrontal cortex (DLPFC), area 46. Research suggests that this cortical activation of DLPFC happens about 150 milliseconds after the onset of a memory stimulus^{2,20,31}. For example, Tomberg²⁹ studied the event-related potentials (ERP) of a working memory task where a subject tracked a target finger stimulus. This stimulus caused a DLPFC negative ERP which occurred at 140 ms. This negative ERP was preceded by a positive ERP located at posterior parietal cortex (area 7). That positive ERP occurred at 100 ms. These findings demonstrate that initial DLPFC activation occurs about 150 ms after the onset of a recognition task stimulus. They also demonstrate that this activation is preceded by an activation of posterior parietal cortex (area 7). Posterior parietal cortex is part of the dorsal stream, see FIG. 1.

The next hippocampal/DLPFC ERPs are the N2-P3 phenomena. Novelty detection ERPs, P3a occur at about 300 ms in lateral prefrontal cortex as well as other cortices²⁴. Independent components analysis (ICA) shows that the target detection ERPs, P3b contribute to the amplitudes of the P3a ERPs¹⁰. Cat studies show that the most ample source of the N2-P3 phenomena is the hippocampus^{3,4}. Additionally, ERP studies of word recognition impairment in schizophrenics show that this impairment begins about 200-300 ms (N2-P3) after stimulus onset¹⁸. These findings demonstrate that there is significant hippocampal/DLPFC activity for delayed recognition between about 150 ms and 300 ms.

These findings allowed us to minimize the data used. We only used data congruent with the task of delayed recognition. To monitor the early part of a delayed recognition task, we examined the data taken from the electrodes which are positioned above posterior parietal cortex (area 7) of each brain hemisphere. These are the electrodes P3 and P4. We examined the data recorded by these electrodes for the first 150 ms after the onset of a delayed recognition stimulus. We also examined the data recorded by EEG electrodes located above the DLPFC of each brain hemisphere. The two electrodes used were additional to standard 10-20 electrodes; T7Fp1 and T8Fp2. T7Fp1 is located in the center of the triangle formed by electrodes Fp1, F3 & F7 and T8Fp2 is located in the center of the electrodes Fp2, F4 & F8 (on the upper edge of each temple). The data examined from these two electrodes were those data that occurred between 151 ms and 300 ms after the onset of the delayed recognition stimulus.

### Quantitative Methods

Quantitative Methods have Four Steps.

1. Collect DLPFC and Posterior Parietal Data

Two basic data sets were collected; the posterior parietal cortical data set (0-150 ms, P3 & P4 electrodes) and the DLPFC cortical data set (151-300 ms, T7Fp1 & T8Fp2 electrodes).

2. Compute a Quantitative Measure of the EEG Activity, Preferably a qEEG Measure which Corresponds to an Informational Measure.

Perform this computation for the electrodes which lie on the scalp above DLPFC and those which lie on the scalp above posterior parietal cortex. In this case, I used the method described in Patent Pending Ser. No. 60/529,944, “A method for Measuring Information which has an Unknown Representation.”

3. Compute the Ratio of DLPFC ^{qEEG} to Posterior Parietal ^{qEEG}.

We compared the values of ^{qEEG} for the anterior EEG data to the posterior EEG data by computing the ratio:

$$qEEG\_Ratio = \frac{{qEEG}_{DLPFC}}{{qEEG}_{PosteriorParietal}}$$

Here, ^{qEEG}^{_{—}}^{Ratio} is the ratio, ^{qEEG}^{_{PosteriorParietal}} is the measure for the posterior parietal electrodes and ^{qEEG}^{_{DLPFC}} is the measure for the DLPFC electrodes. This ratio was computed twice; once for the delayed recognition faces task, and once for the delayed recognition objects task. These two ratios were used to compute an average ratio.

4. Data Accuracy.

To achieve accurate results, the EEG data must be free of confounds. For this reason, we excluded eye blinks, muscle movements and 60 Hz interference (caused by electrical coupling with 60 Hz AC sources) during the first 300 ms after the onset of the stimulus. Otherwise, the data were unfiltered. Subjects needed to complete at least 10 delayed recognition trials in order to compute a reliable qEEG measure whose standard deviation is 2% or less of its value.

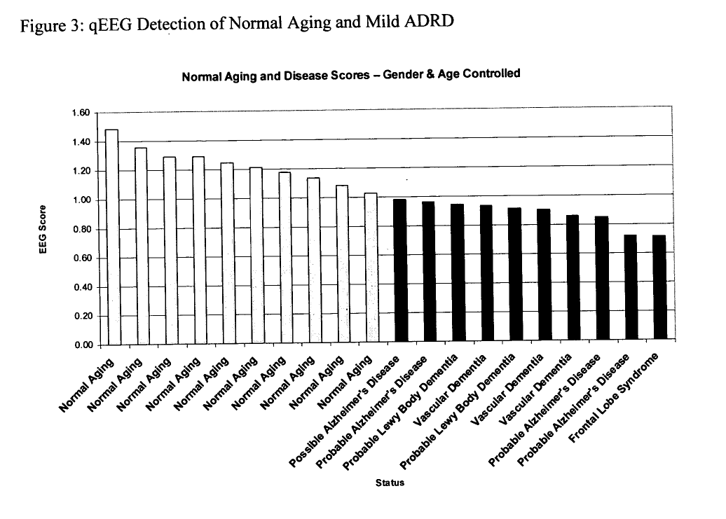

This method, applied to 20 individuals controlled for age, gender and cholinesterase inhibitor treatment yielded the results given in FIG. 3. Note the wholly unanticipated result that the dividing point between normal aging and ADRD is at 1.00±0.02. Thus, this test not only uses a person as their own baseline for comparison, no further comparison is needed. A person with a scored above 1.00±0.02 is normal aging, a person below, has ADRD. This method also yielded the following results:

1. Detecting Mild Cognitive Impairment (MCI) and Mild Dementia ADRD with an accuracy of 92%. The sensitivity was 88%; the specificity was 94%. 13 subjects had MCI ADRD, 3 had mild dementia ADRD, 32 were normal aging. There were 2 false negative subjects and 2 false positive subjects.

The qEEG method computes a predictive value, the “qEEG ratio,” the ratio of DLPFC activity to posterior parietal activity. Subjects' qEEG ratios ranged from a lowest value of 0.71 to a highest value of 1.54. The average qEEG ratio for normal aging subjects was 1.20±0.06, 95% Confidence Interval (CI); the average value for ADRD subjects was 0.92±0.08, 95% CI. These qEEG ratios had a high negative correlation with Functional Assessment Staging²² (FAST) scores; ρ=−0.71±0.02, 95% CI, p<0.001 (t-test based on the Fisher transform). We compared the accuracy of this qEEG method to a standard qEEG technique; computing the relative theta power^{1,5}. The theta power produced a sensitivity, specificity, and accuracy of 75%. Statistically, the present method is more accurate; p<0.005 (binomial test). We also compared the present method to CERAD memory tests. CERAD tests produced a sensitivity of 50%, a specificity of 70% and a total accuracy of 63%. The present method is more accurate; p<0.001.

### Operation—Additional Embodiments

1. Detection of preclinical ADRD. The qEEG method detects very early ADRD. MRI studies show that hippocampal atrophy appears early in AD¹⁵. Eleven of the ADRD subjects had MRI exams. Of these eleven, four had no hippocampal atrophy. The qEEG method detected all four of these subjects. We expect to see false positive subjects; however the two false positive subjects described in part 1 may actually be preclinical ADRD. Both were relatively young. One was in their mid 40s; the other was in their earlier 50s. Both subjects had parents with Alzheimer's Disease. Both had objective cognitive deficits. One subject had left frontal cognitive impairment (verbal fluency); the other had dorsolateral-prefrontal cortical (DLPFC) cognitive impairment (working memory).

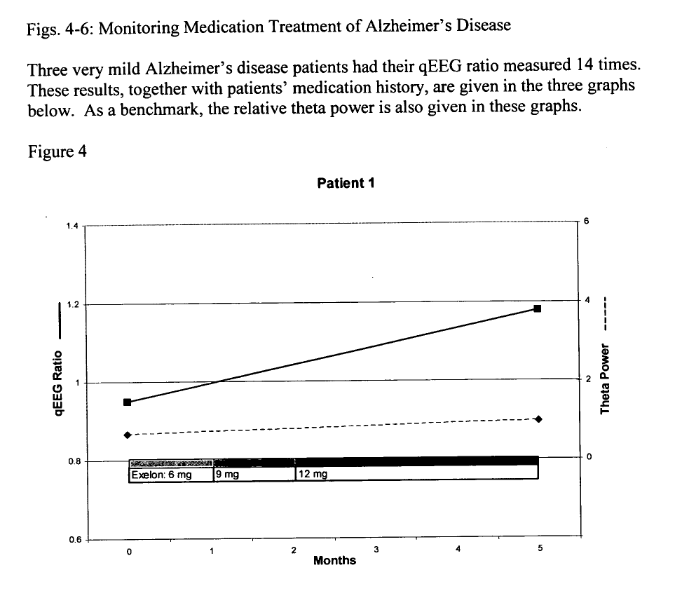

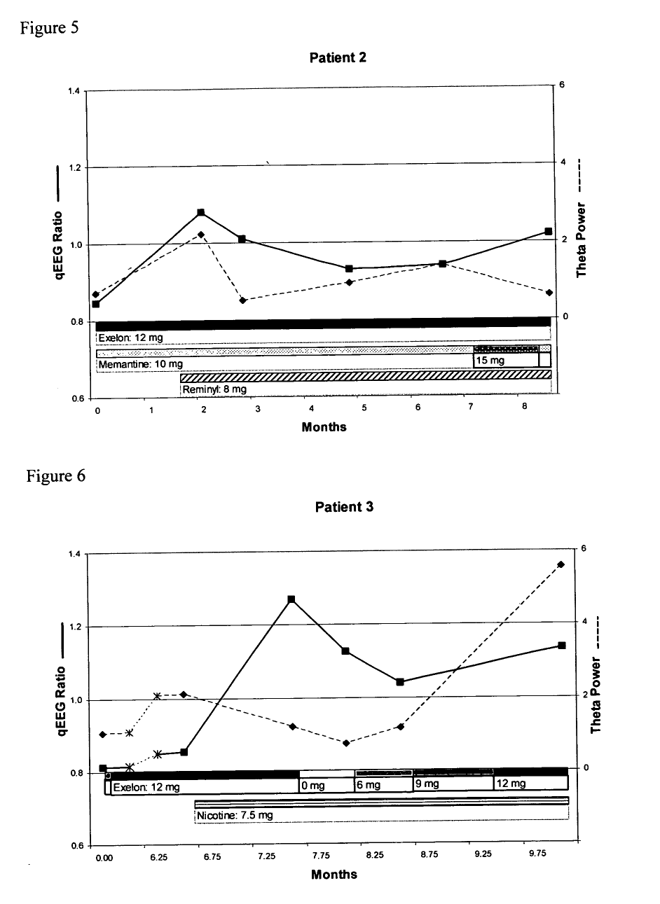

2. Measuring individual treatment effects. The qEEG method is sensitive to small changes in neurophysiology. Three early AD subjects had their EEG recorded on 14 occasions. These 14 measurements yielded 11 measures of qEEG change. These changes accurately reflected individual changes in medication on 10 of 11 occasions (91%), p<0.005 (binomial test). We expect to see false negative subjects; however the two false negative subjects described in part 1 may actually be the result of a treatment effect. Both subjects had begun cholinesterase inhibitor treatment. Both had made the subjective report that their memory had improved. Their CERAD memory test scores were normal. Three graphs which show the 14 qEEG measurements and medication information are in the FIGS. 4-6.

3. Measuring Delayed Recognition memory. The qEEG method analyzes EEG data taken while a subject performs Delayed Recognition memory tasks. If the method is specific to Delayed Recognition memory, then data recorded during a non-memory task should not detect ADRD. We applied the qEEG method to data taken from a non-memory task, the perception of structure from motion (SFM). An optimal cutoff value yielded a sensitivity of 63%, a specificity of 56% and a total accuracy of 58%. This result is not significantly different from chance. Subjects' qEEG ratios for the SFM task ranged from a lowest value of 0.53 to a highest value of 1.30. The average qEEG ratio for normal aging subjects was 0.90±0.06, 95% CI; the average value for ADRD subjects was 0.86±0.08, 95% CI. These qEEG ratios had a non-significant correlation with FAST scores; ρ=−0.06±0.05, 95% CI.

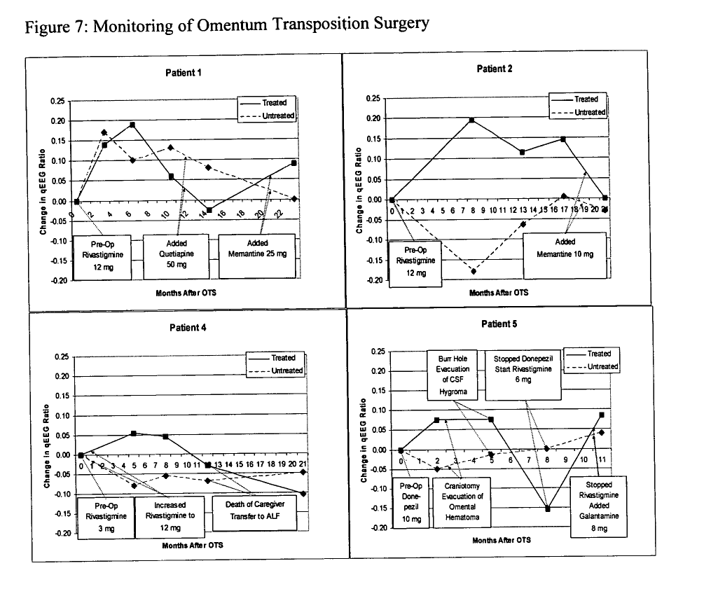

4. Measuring and distinguishing surgical effects applied to a single brain hemisphere. The qEEG method is sensitive to changes in a single brain hemisphere. Four individuals with refractory Alzheimer's disease had omentum transposition surgery (OTS). This surgery transposes the patient's omentum onto one brain hemisphere. The overall effect of OTS, was measured by a “total qEEG ratio.” Mini Mental Status Exam (MMSE) scores correlate with the total qEEG measures, ρ=0.54±0.06, p<0.006, (t-test based on the Fisher transform, 95% confidence interval). The individual correlations of patients 1, 2, 4 and 5 are 67%, 28%, 64% and 67% respectively (patient 3 did not participate in EEG). The individual with the low correlation, 28%, had violated medical protocol. Separate brain hemispheric effects were also measured. These measurements are graphed in FIG. 7.

### Operation—Annother Additional Embodiment

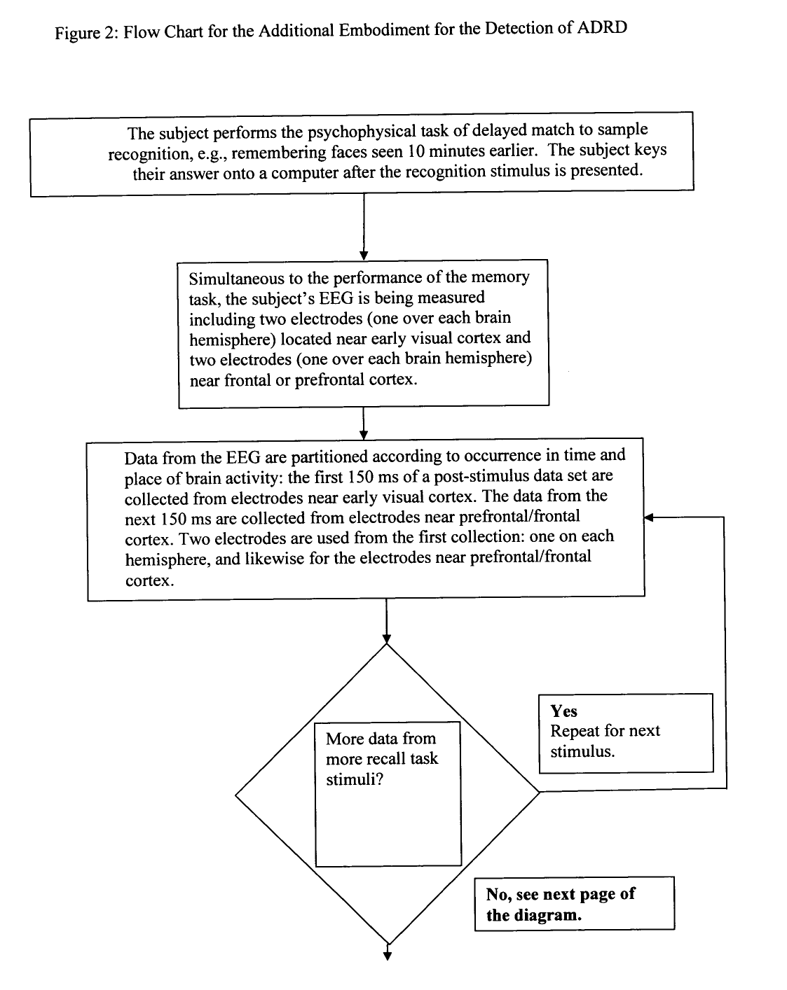

Turning to FIG. 2 another embodiment is discussed. Here, the user can use a method of brain attribute measurement including, but not limited to Electroencephalogram, Positron Emission Tomography, Magnetic Resonance Imaging, functional Magnetic Resonance Imaging, Computer Aided Tomography, SPECT etc.

Information or an informational type of measure or an approximation thereof is computed from the obtained data using a method such as that described in the provisional patent, “A method for measuring information which has an unknown representation,” inventor: Robert Sneddon. Such information or informational type of measure or an approximation thereof is computed at time intervals and brain and/or cranial and/or head points which are most appropriate for the function being measured.

The person being examined may, but does not have to, be performing a task, e.g., an appropriate psychophysical task, which engenders the appropriate brain systems' activity.

Information or an informational type of measure or an approximation thereof amounts and/or change is computed at different times and different places appropriate to the task and/or brain function being examined. This knowledge is then used to differentiate between possible disorders and/or diseases and/or functions. That differentiation can, but does not have to use a criterion for differentiation of different disorders and/or diseases and/or functions based on an amount or change in information or information-like measure between different times and different brain areas.

Whereas the present invention has been described in particular relation to the drawings attached hereto, it should be understood that other and further modifications apart from those shown or suggested herein, may be made within the scope and spirit of the present invention.

References

1. Anderer P, Saletu B, Kloppel B, et al: Discrimination between demented patients and normals based on topographic EEG slow wave activity: comparison between z statistics, discriminant analysis and artificial neural network classifiers. Electroencephalogr Clin Neurophysiol 91:108-117, 1994
2. Barlow J S: The electroencephalogram: its patterns and origins. Cambridge, Mass.: MIT Press, 1993
3. Basar-Eroglu C, Basar E, Schmielau F: P300 in freely moving cats with intracranial electrodes. Int J Neurosci 60:215-226, 1991
4. Basar-Eroglu C, Schmielau F, Schramm U, et al: P300 response of hippocampus analyzed by means of multielectrodes in cats. Int J Neurosci 60:239-248, 1991
5. Belouchrani A: Discrimination of Alzheimer's disease and normal aging by EEG data. Electroencephalography and clinical neurophysiology 103:241-248, 1997
6. Bunsey M, Eichenbaum H: Conservation of hippocampal memory function in rats and humans. Nature 379:255-257, 1996
7. Cabeza R, Dolcos F, Graham R, et al: Similarities and differences in the neural correlates of episodic memory retrieval and working memory. Neuroimage 16:317-330, 2002
8. Courtney S M, Petit L, Haxby J V, et al: The role of prefrontal cortex in working memory: examining the contents of consciousness. Philos Trans R Soc Lond B Biol Sci 353:1819-1828, 1998
9. Debanne D, Guerineau N C, Gahwiler B H, et al: Action-potential propagation gated by an axonal I-A-like K+ conductance in hippocampus. Nature 389:286-289, 1997
10. Debener S, Makeig S, Delorme A, et al: Functional significance of the novelty P3 event—related potential as revealed by independent component analysis, in Neurosciences 2004. San Diego: Society for Neuroscience, 2004
11. Durand G M, Kovalchuk Y, Konnerth A: Long-term potentiation and functional synapse induction in developing hippocampus. Nature 381:71-75, 1996
12. Elbert T, Schauer M: Burnt into memory. Nature 419:883-883, 2002
13. Fallon J, Opole I, Potklin S: The Neuroanatomy of Schizophrenia: at the Molecular, Cellular, and Systems Levels. Clinical Neurological Research 3:77-107, 2003
14. Ferreira C T, Verin M, Pillon B, et al: Spatio-temporal working memory and frontal lesions in man. Cortex 34:83-98, 1998
15. Fox N C, Warrington E K, Freeborough P A, et al: Presymptomatic hippocampal atrophy in Alzheimer's disease. A longitudinal MRI study. Brain 119 (Pt 6):2001-2007, 1996
16. Ishai A, Ungerleider L G, Haxby J V: Distributed neural systems for the generation of visual images. Neuron 28:979-990, 2000
17. Ishai A, Ungerleider L G, Martin A, et al: The representation of objects in the human occipital and temporal cortex. J Cogn Neurosci 12 Suppl 2:35-51, 2000
18. Kayser J, Bruder G E, Friedman D, et al: Brain event-related potentials (ERPs) in schizophrenia during a word recognition memory task. Int J Psychophysiol 34:249-265, 1999
19. Knight R T, Nakada T: Cortico-limbic circuits and novelty: a review of EEG and blood flow data. Rev Neurosci 9:57-70, 1998
20. Linkenkaer-Hansen K, Palva J M, Sams M, et al: Face-selective processing in human extrastriate cortex around 120 ms after stimulus onset revealed by magneto- and electroencephalography. Neuroscience Letters 253:147-150, 1998
21. Poldrack R A, Clark J, Pare-Blagoev E J, et al: Interactive memory systems in the human brain. Nature 414:546-550, 2001
22. Reisberg B: Functional assessment staging (FAST). Psychopharmacol Bull 24:653-659, 1988
23. Rugg M D, Fletcher P C, Chua P M, et al: The role of the prefrontal cortex in recognition memory and memory for source: an fMRI study. Neuroimage 10:520-529, 1999
24. Soltani M, Knight R T: Neural origins of the P300. Crit Rev Neurobiol 14:199-224, 2000
25. Squire L R: Memory from A to Z. Nature 423:119-119, 2003
26. Squire L R: Searching for memory: The brain, the mind, and the past—Schacter, D L. Nature 382:503-504, 1996
27. Squire L R: The Seven Sins of Memory: How the mind forgets and remembers. Nature 413:571-572, 2001
28. Stevens C F: Strengths and weaknesses in memory. Nature 381:471-472, 1996
29. Tomberg C: Cognitive N140 electrogenesis and concomitant 40 Hz synchronization in mid-dorsolateral prefrontal cortex (area 46) identified in non-averaged human brain potentials. Neurosci Lett 266:141-144, 1999
30. Wagner A D, Desmond J E, Glover G H, et al: Prefrontal cortex and recognition memory. Functional-MRI evidence for context-dependent retrieval processes. Brain 121 (Pt 10):1985-2002, 1998
31. Watanabe S, Kakigi R, Koyama S, et al: Human face perception traced by magneto- and electro-encephalography. Cognitive Brain Research 8:125-142, 1999

A method to examine neurological function consisting of

1. A method for detecting brain function which comprises the steps of:
a. Measuring an individual's brain state with a device designed for such measurements, while they are performing a plurality of psychological tasks or behavioral tasks, each of the same kind
b. generating two or more data sets from the measuring device's data, where each data set contains data from a plurality of the individual tasks, but each data set differs in some manner from the other
c. a means for comparing an aspect of one or more sets of said data with an aspect of one or more of said different data sets such that if the aspect in one or more data sets is different in some manner from said one or more different data sets, the person has a specific type of neurological function

2. The method of claim 1 where said data sets differ because they are taken at different time intervals within the time course of the individual tasks

3. The method of claim 1 where said data subsets differ because they contain data taken from a plurality of the individual tasks which refer to different aspects of the brain

4. The method of claim 1 where said data subsets differ because they contain data taken from a plurality of the individual tasks which refer to different aspects of the brain and are taken from different time intervals

5. The method of claim 4 where said device for measuring a brain state is an electroencephalography (EEG) machine.

6. The method of claim 5 where said task requires the use of a specific neurological function

7. The method of claim 6 where the specific neurological function is delayed recognition memory and the task includes a recognition memory stimulus

8. The method of claim 7 where said EEG data sets differ because one set is taken from electrodes located over the posterior parietal cortex during a time interval substantially similar to the first 150 milliseconds (ms) after the onset of a delayed recognition stimulus and a second data set is taken from electrodes located over the dorsolateral prefrontal cortex and taken during a time interval substantially similar to the second 150 ms after the onset of the recognition stimulus

9. The method of claim 8 where the two data sets are compared to each other by measuring there activity and then finding which activity is greater so that if the first data set's activity is greater than or equal to the seconds, the person have memory impairment related to a neurological disease or disorder

10. The method of claim 9 where the type of activity measurement is substantially related to the variability of the data.

11. The method of claim 10 where the type of activity measurement is substantially related to informational properties.

12. The method of claim 11 where the informational activity measurement is performed by the method described in patent application Ser. No. 60/529,944, “A method for Measuring Information which has an Unknown Representation.”

13. The method of claim 1 where the psychological tasks performed are used to elicit emotional responses

14. The method of claim 1 where the psychological tasks performed are used to check to see if a person is telling the truth or not

15. The method of claim 6 where the neurological function measured is a treatment effect.

16. The method of claim 6 where the neurological function measured is the effect of surgery.

17. The method of claim 6 where the neurological function measured is a predisposition to ADRD.

18. The method of claim 6 which uses specific neurological tasks which are specific to different neurological functions for the differential diagnosis of neurological disorders and diseases

19. The method of claim 6 which uses specific neurological tasks which are specific to different neurological functions for the detection of neurological disorders and diseases

20. The method of claim 6 which uses specific neurological tasks which are specific to different neurological functions for the monitoring of neurological disorders and diseases
